(19) United States
(12) Patent Application Publication
(10) Pub. No.: US 2019/0359621 A1
(43) Pub. Date: Nov. 28, 2019

(54) BICYCLIC UREA COMPOUNDS AND THEIR USE AS SOLVENT

(71) Applicant: BASF SE, Ludwigshafen am Rhein (DE)

(72) Inventors: Nicolas MARION, Saint-Louis (FR); Ralph BUSCH, Ludwigshafen (DE); Artur KOZICKI, Ludwigshafen (DE); Ulrich KARL, Ludwigshafen (DE); Alexander PANCHENKO, Ludwigshafen (DE); Johann-Peter MELDER, Ludwigshafen (DE)

(73) Assignee: BASF SE, Ludwigshafen am Rhein (DE)

(21) Appl. No.: 16/481,105

(22) PCT Filed: Feb. 9, 2018
(86) PCT No.: PCT/EP2018/053279

§ 371 Date: Jul. 26, 2019

(30) Foreign Application Priority Data
Feb. 15, 2017 (EP) 17156273.9

(51) Int. Cl. C07D 487/08; C07D 235/06

(52) U.S. Cl. CPC C07D 487/08; C07D 235/06

(57) ABSTRACT

A compound of formula (I) of formula (II) or of formula (III) wherein R¹ and R² independently from each other are a C1- to C8-alkyl group and R³ to R¹² independently from each other are a hydrogen or a C1- to C4-alkyl group.

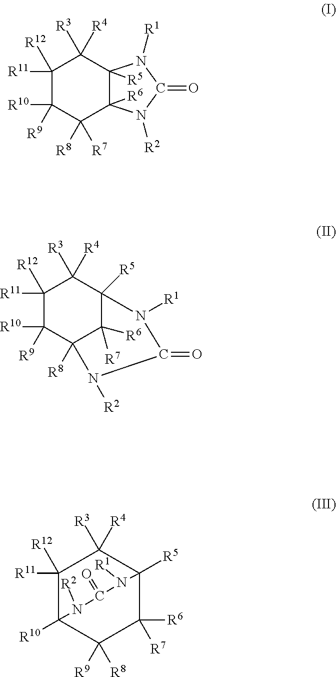

Object of the present invention is a compound of formula I

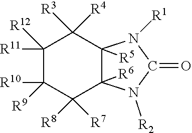

of formula II

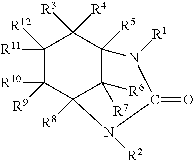

or of formula III

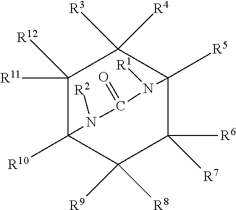

wherein R¹ and R² independently from each other are a C1- to C8-alkyl group and R³ to R¹² independently from each other are a hydrogen or a C1- to C4-alkyl group.

Liquid organic compounds are often used as solvents, working fluids, heat transfer fluids or as cleaning agents. For many applications polar liquid compounds are required. Compounds which have a certain polarity may, for example, solve polar organic polymers. Often it is desired that such polar compounds are aprotic. Aprotic compounds do not have hydrogen that easily dissociates as cation. A well-known polar aprotic solvent is N-methyl pyrrolidone.

In industry there is always a demand for new liquid organic compounds that may have satisfying or even improved properties and are reasonable alternatives, for example, as solvents. For example, WO 2015/024824 and WO 2015/197380 disclose the use of N-formyl morpholine as solvent for polyamide-imide and polyvinylidene fluoride and PCT/EP2016/069787 discloses the use of N-formyl pyrrolidine as solvent for polyimides.

Earlier EP patent application number 16173915.6 (INV 150243) discloses the formation of urea adducts in order to separate cis- and trans-4-methylcyclohexan-1,3 diamine.

A suitable solvent for polymers should in particular allow the preparation of solutions with high polymer content.

It was an object of the present invention to provide alternative liquid chemical compounds that are suitable for many technical applications; in particular, as solvents, working fluids, heat transfer fluids or as cleaning agents. The process for the production of such alternative solvents should be easy to perform and of low costs.

Accordingly, the compounds according to formula I, II and III and their use as solvent, working fluid, heat transfer fluid or as cleaning agent have been found.

To the compound

R¹ and R² in formula I, II or III independently from each other are a C1- to C8-alkyl group. Preferably, R¹ and R² in formula I, II or III independently from each other are a C1- to C4-alkyl group such as a methyl, ethyl, iso-propyl or n-butyl group.

In a most preferred embodiment of the invention both, R¹ and R² are a methyl group.

R³ to R¹² independently from each other are a hydrogen or a C1- to C4-alkyl group.

Preferably, most of R3 to R12 are a hydrogen atom. In particular, at maximum three, preferably at maximum two of R3 to R12 in formula I, II or III are a C1- to C4 alkyl group and all the other R3 to R12 are a hydrogen atom.

In a most preferred embodiment of the present invention the compound is a compound of formula II or a mixture of compounds of formula II.

A preferred compound of formula II is the compound of formula IIa

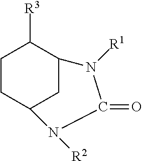

or the compound of formula IIb

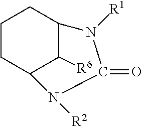

wherein R¹, R², R³ and R⁶ have the meanings and preferred meanings as defined above. Any of R³ to R¹² not shown in formula IIa and IIb are a hydrogen atom.

A more preferred compound of formula II is a mixture of a compound of formula IIa and IIb.

A most preferred compound of formula II is a mixture of a compound of formula IIa and IIb wherein R³ and R⁶ are a methyl group and R¹ and R² have the meaning as defined above and are in particular a methyl group.

Preferably, the mixture of compounds IIa and IIb comprises 50 to 95% by weight of IIa and 5 to 50% by weight of IIb, based on the total weight of IIa+IIb. More preferred is a mixture comprising 70 to 90% by weight of IIa and 10 to 30% by weight of IIb, based on the total weight of IIa+IIb. Most preferred is a mixture comprising 75 to 85% by weight of IIa and 15 to 25% by weight of IIb, based on the total weight of IIa+IIb.

### Synthesis of the Compound

Compounds of formula I, II and II may be synthesized by a two steps process.

In a first step urea is reacted with a cyclohexyl compound comprising a cyclohexyl ring system wherein two of the six carbon atoms of the ring system are substituted by a primary amino group and the other carbon atoms of the ring system may optionally be substituted by alkyl groups according to the definition of R³ to R¹². The reaction is known to the man skilled in the art and is described for example by J. G. Michels in Journal of Organic Chemistry 1960, 25, 2246-2247.

Compound of formula I requires a cyclohexyl compound which is substituted by a two primary amino groups in 1, 2 position to the ring system.

Compound of formula II requires a cyclohexyl compound which is substituted by a two primary amino groups in 1, 3 position to the ring system.

Compound of formula III requires a cyclohexyl compound which is substituted by a two primary amino groups in 1, 4 position to the ring system.

In case of a mixture of compounds IIa and IIb a corresponding mixture of two isomeric cyclohexyl compounds should preferably be used as starting material. Such a mixture is easily obtained by hydrogenation of a mixture of 2,4 and 2,6 diamino toluene according to the following equation

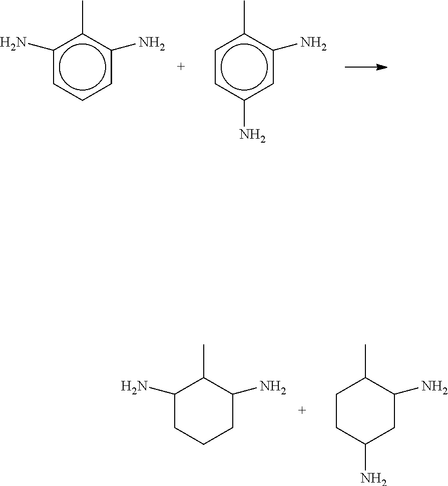

The cyclohexyl compound or mixture of cyclohexyl compounds may be reacted with urea as known in the art. The reaction may be performed under reduced, normal or elevated pressure. Usually it is performed at normal pressure (1 bar). Preferably, the content of oxygen should be low. Hence the reaction is preferably performed in an atmosphere of an inert gas such as nitrogen. Preferably, the reaction is performed at elevated temperature, preferably at a temperature of 100 to 250° C., in particular at a temperature from 150 to 220° C. In the reaction ammonia is set free and is preferably continuously removed.

In a second step the two secondary amino groups are alkylated. The alkylation may be performed according to any method know in the art. In a preferred embodiment formaldehyde or compounds which set free formaldehyde, such as paraformaldehyde, are used as methylation agents. Alkylation may preferably be performed in presence of an acid, such as, for example, formic acid. The alkylation reaction may be performed under reduced, normal or elevated pressure. Usually it is performed at normal pressure (1 bar). Preferably, the reaction is performed at elevated temperature, preferably at a temperature of 40 to 150° C., in particular at a temperature from 80 to 140° C.

The final product is obtained in high yield and high selectivity.

To the use of the compound

The above preferred embodiments regarding compound I, II, and III and in particular all above preferred embodiments of the compound of formula II apply to the use described below.

The compound of formula I, II or III may be used as solvent, working fluid, heat transfer fluid or as cleaning agent. Preferably, the compound of formula I, II or III is used as a solvent. In a more preferred embodiment the compound is used as solvent for polymers. Use as solvent for polymers shall include the dissolution of polymers in the solvent or the synthesis of polymers in the solvent.

Most preferred is the use of compound of formula II and in particular of mixtures of compounds IIa and IIb as solvent for polymers.

In a preferred embodiment the polymers are selected from sulfone polymers, polyamide, polyimide, polyamidimide, polyester and halogen substituted polymers.

A sulfone polymer comprises —SO2- units in the polymer, preferably in the main chain of the polymer.

Preferably, the sulfone polymer comprises at least 0.02 mol —SO2- units, in particular at least 0.05 mol —SO2- units per 100 grams (g) of polymer. More preferred is a sulfone polymer comprising at least 0.1 mol —SO2- units per 100 g of polymer. Most preferred is a sulfone polymer comprising at least 0.15 mol SO2- units, in particular at least 0.2 mol —SO2- units per 100 g of polymer.

Usually a sulfone polymer does comprise at maximum 2 mols —SO2- units, in particular at maximum 1.5 mols —SO2- units per 100 grams (g) of polymer. More preferred is a sulfone polymer comprising at maximum 1 mol —SO2- units per 100 grams of polymer. Most preferred is a sulfone polymer comprising at maximum 0.5 —SO2- units per 100 grams of polymer.

Preferably, the sulfone polymer comprises aromatic groups, shortly referred to as an aromatic sulfone polymer.

In a preferred embodiment, the sulfone polymer is an aromatic sulfone polymer, which consists to at least 20% by, in particular to at least 30% weight of aromatic carbon atoms. An aromatic carbon atom is a carbon atom, which is part of an aromatic ring system.

More preferred is an aromatic sulfone polymer, which consists to at least 40% by weight, in particular to at least 45% by weight of aromatic carbon atoms.

Most preferred is an aromatic sulfone polymer, which consists to at least 50% by weight, in particular to at least 55% by weight of aromatic carbon atoms.

Preferably, the sulfone polymer may comprise aromatic groups that are selected from 1,4-phenylen, 1,3-phenylene, 1,2-phenylene, 4,4′-biphenylene, 1,4-naphthylene, or 3-chloro-1,4-phenylene.

The aromatic groups may be linked by, for example, units selected from —SO2-, —SO—, —S—, —O—, —CH2-, —C(CH3)2.

In a preferred embodiment, the sulfone polymer consists to at least 80% by weight, more preferably to at least about 90% by weight and most preferably to at least 95, respectively at least 98% by weight of groups selected from the above aromatic groups and linking groups.

Examples of most preferred sulfone polymers are polyethersulfone, polysulfone, polyphenylsulfone.

Polyethersulfone is a polymer of formula IV

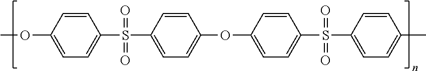

which is, for example, available from BASF under the trade name Ultrason® E.

Polysulfone is a polymer of Formula V

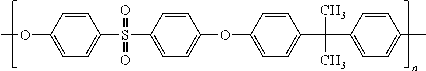

which is, for example, available from BASF under the trade name Ultrason® S.

Polyphenylsulfone is a polymer of formula VI

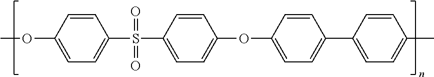

which is, for example, available from BASF under the trade name Ultrason® P.

Most preferred polymers are selected from polyethersulfone, polysulfone, polyphenylsulfone and poly vinyliden fluoride.

In a particularly preferred embodiment the polymer is a sulfone polymer and is in particular selected from polyethersulfone, polysulfone, polyphenylsulfone.

The use of the compound of formula I, II or III results as solvent for polymers results in polymer solutions comprising the respective polymer and the compound as solvent.

The polymer solutions may comprise other solvents besides compounds of formula I, II or III. Other solvents might be, for example, N-methylpyrrolidone (NMP), N-ethylpyrrolidon (NEP), dimethylacetamide (DMAc), dimethylformamide (DMF), dimethylacrylamide (DMAD), dimethylsulfoxide (DMSO) or alkylencarbonates as such as in particular propylene carbonate.

In a preferred embodiment of the invention at least 30% of all solvents of the polymer solution, more preferred at least 50% of all solvents of the polymer solution and most preferred at least 70% of all solvents of the polymer solution are compounds of formula I, II or III. In a particular embodiment of the invention, the compound of formula I, II or II is the only solvent in the polymer solution.

At low temperatures the solubility of the polymer in the solvent might be limited. However, higher amounts of the polymer will become fully dissolved at higher temperatures. Hence polymer solutions comprising 10 to 50% by weight of polymer are obtainable at temperatures of the polymer solution of at least 60° C. Polymer solutions comprising 20 to 50% by weight of polymer are obtainable at temperatures of the polymer solution of at least 80° C., respectively of at least 100° C. The temperature of the polymer solutions is usually not higher than 200° C. The polymer solutions may have various technical applications, one preferred technical application of polymer solutions is the use as coating material. Such technical applications, in particular coating processes, are performed at higher temperature as of 60 to 200° C. Hence the solubility of the polymer in the solvent at such temperatures is required.

### EXAMPLES

Abbreviations and compounds used in the examples:
PPS Polyphenylsulfone of formula VI (Poly(oxy-1,4-phenylene-1,4-sulphonylphenylene), obtained from Aldrich
PES Polyethersulfone of formula IV, Ultrason® E 6020 of BASF
PS Polysulfone of formula V; Mw=35000, Mn=16000, obtained from Aldrich
PVFD Polyvinylidendifluoride, Mw=534000, obtained from Aldrich
NMP: N-methyl pyrrolidone
NFP: N-formyl pyrrolidine

The methylated diamino methyl cyclohexane urea used was a mixture composed of ca 80% by weight of compound of formula IIa and 20% by weight of compound of formula IIb, whereby all of R1, R2, R3 and R6 are a methyl group.

### Example 1: Preparation of Methylated Diamino Methyl Cyclohexan Urea

As starting material a diamino methyl cyclohexane mixture of 80% by weight of 1-methyl, 2,4 diamino cyclohexane and of 20% by weight of 1-methyl-, 2,6 diamino cyclohexane, obtained by hydrogenation of the corresponding mixture of 2,4 and 2,6 diamino toluene, has been used.

In a first step 1385 g (10.8 mol) of the diamino methyl cyclohexane mixture and 120 g (2 mol) of urea have been filled in a batch reactor. The reactor was kept at 40° C. and evacuated in order to remove oxygen. When a pressure of 50 mbar was reached, the reactor was refilled with nitrogen and adjusted to normal pressure (1 bar). Thereafter temperature was increased in 6 hours from 40 to 200° C., kept 3 hours at 200° C. and then cooled down in 2 hours from 200 to 20° C. Ammonia formed was steadily removed in a stream of nitrogen of 100 liters per hour.

The obtained product was separated from fluid components by a suction filter and was washed with xylene.

The product was the urea of the diamino methyl cyclohexane mixture wherein the nitrogen atoms are substituted by hydrogen. The yield of the urea of diamino methyl cyclohexane was 82.9% by weight based on urea (determined by gas chromatography and determination of the area of the relevant peaks).

In a second step the two nitrogen atoms of the urea of methyl diamino cyclohexane were methylated in order to obtain the compounds of formula IIa and IIb with R¹, R², R³ and R⁶ being methyl groups.

Alkylation was performed with paraformaldehyde as alkylating agent in the presence of formic acid at normal pressure. 588.1 g (3.16 mol) of the urea of the diamino methyl cyclohexane and 242.1 g (7.43 mol) of paraformaldehyde and 567.9 g (11.38 mol) of formic acid were filled into the reactor. at room temperature (21° C.) within 3 hours the reactor was heated to 101° C. and kept between 101 and 110° C. for 16 hours. Thereafter the reactor was cooled down to 25° C.

In order to remove excess formic acid 666 g (5.94 mol) of aqueous potassium hydroxide solution were added over a period of one hour and the pH value was 8.8. Two phases were formed. The lower phase was aqueous potassium formiate, the upper phase was an organic liquid. The upper phase was separated from the lower phase and distilled. The yield of methylated diamino methyl cyclohexane urea was 94% by weight based on the urea of the diamino methyl cyclohexane.

### Example 2

### Use of the Methylated Diamino Methyl Cyclohexane Urea as Solvent for Polymers.

Different polymers were added to NMP, NFP (both for comparison) and to methylated diamino methyl cyclohexane urea prepared according to example 1 (in the table for short: alkylated urea). It was determined how many parts by weight of polymer could be solved in 100 parts by weight of solvent at different temperatures. Results are listed in the table below. The numbers in the table represent the parts by weight of a polymer which could at maximum be solved at a given temperature and gave a clear solution. The solution turned turbide after further addition of polymer.

TABLE

| | PES | | | | | | PS | | |
|---|---|---|---|---|---|---|---|---|---|
| | | | alkylated | PPS | | | | | alkylated |
| | NMP | NFP | urea | NMP | NFP | Urea | NMP | NFP | urea |
| 20° C. | 15 | 0 | 0 | 0 | 0 | 0 | 0 | 0 | 0 |
| 40° C. | 15 | 0 | 0 | 5 | 0 | 0 | 5 | 0 | 0 |
| 60° C. | 25 | 10 | 0 | 10 | 10 | 0 | 10 | 5 | 0 |
| 80° C. | 45 | 35 | 15 | 20 | 20 | 0 | 15 | 10 | 20 |
| 100° C. | 65 | 65 | 20 | 25 | 30 | 2 | 25 | 15 | 26 |
| 120° C. | 75 | v | 32 | 30 | 35 | 43 | 45 | 25 | 34 |

| | PVFD | | |
|---|---|---|---|
| | | | alkylated |
| | NMP | NFP | urea |
| 20° C. | 0 | 0 | 0 |
| 40° C. | 0 | 0 | 0 |
| 60° C. | 5 | 15 | 2 |
| 80° C. | 20 | 35 | 8 |
| 100° C. | 35 | 50 | 16 |
| 120° C. | 55 | 80 | 40 |

1. A compound of formula I 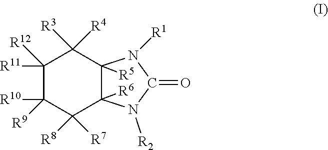
or of formula II 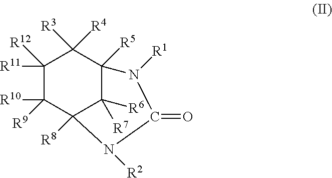
or of formula III 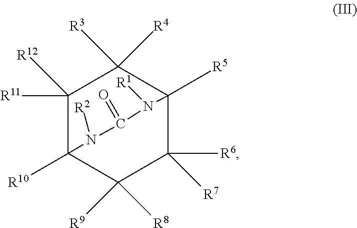
wherein R¹ and R² independently from each other are a C1- to C8-alkyl group and R³ to R¹² independently from each other are hydrogen or a C1- to C4-alkyl group.

2. The compound according to claim 1, wherein at maximum three of R³ to R¹² in formula I, II or III are a C1- to C4 alkyl group.

3. The compound according to claim 1, wherein the compound is a compound of formula II or a mixture of compounds of formula II.

4. The compound according to claim 1, wherein the compound is a mixture of a compound of formula IIa 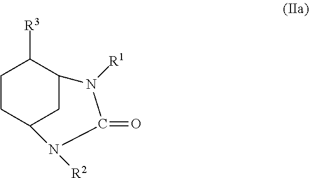
and of a compound of formula IIb

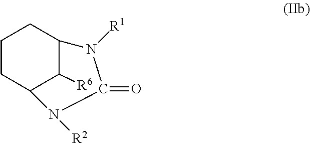

5. A solvent, a working fluid, a heat transfer fluid or a cleaning agent, comprising
the compound according to claim 1.

6. A solvent for a polymer, comprising
the compound according to claim 1.

7. The solvent according to claim 6, wherein the compound is a compound of formula II or a mixture of compounds of formula IIa and formula IIb:

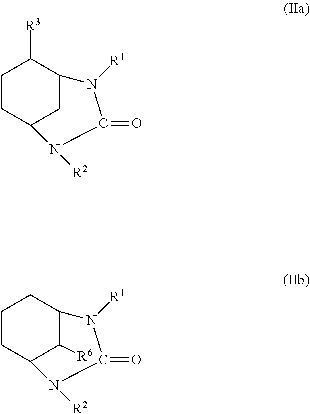

8. The solvent according to claim 6, wherein the polymer is at least one selected from the group consisting of a sulfone polymer, a polyamide, a polyimide, a polyamidimide, a polyester, and a halogen substituted polymer.

9. The solvent according to claim 6, wherein the polymer is at least one selected from the group consisting of a polyethersulfone, a polysulfone, a polyphenylsulfone and a poly vinyliden fluoride.

10. A polymer solution, comprising
the compound according to claim 1 as a solvent.

11. The polymer solution according to claim 10, wherein at least 30% of all solvents of the polymer solution are the compound.
